(19) United States
(12) Patent Application Publication
(10) Pub. No.: US 2026/0108423 A1
(43) Pub. Date: Apr. 23, 2026

(54) Kit Comprising a Vial and a Male Connector

(71) Applicant: UNITHER PHARMACEUTICALS, PARIS (FR)

(72) Inventors: Marc MAURY, ST MEDARD EN JALLES (FR); Michel LE CADRE, COURCY (FR); Cyrille MARECHAL, RIOM (FR)

(21) Appl. No.: 19/111,504

(22) PCT Filed: Sep. 18, 2023
(86) PCT No.: PCT/FR2023/051420

§ 371 Date: Mar. 13, 2025

(30) Foreign Application Priority Data
Sep. 16, 2022 (FR) 2209377

(51) Int. Cl. A61J 1/20; A61J 1/06; A61J 1/14; A61M 5/28; A61M 5/34

(52) U.S. Cl. CPC A61J 1/2055; A61J 1/067; A61J 1/1431; A61J 1/2086; A61M 5/282; A61M 5/347

(57) ABSTRACT

The invention relates to a kit comprising a vial and a male connector (6) comprising: a male part (8) suitable for being inserted in a female connector of the vial, the male part having an outer surface (12) extending around an axis (X) and tapering towards a free end (2) of the male part (8); a sleeve (14) extending around and at a distance from the male part (8), the sleeve (14) comprising an internal thread (20) suitable for cooperating with an external thread of the female connector when the male part (6) is inserted in the female connector, the outer surface (12) comprising a first frustoconical portion (12b) forming a first angle (α) with respect to the axis (X), and a second frustoconical portion (12c) extending the first frustoconical portion (12a) towards the free end (2), the second frustoconical portion (12c) forming a second angle (β) with respect to the axis (X), the second angle being less than the first angle (α).

### FIELD OF THE INVENTION

The present disclosure relates to a kit comprising a vial and a male connector.

### PRIOR ART

It is known to use “luer lock” connectors to fluidically connect a first piece of equipment and a second piece of equipment. For this purpose, the first piece of equipment comprises a luer-lock female connector, and the second piece of equipment comprises a luer-lock male connector. Such connectors are used, for example, in needle or syringe devices.

The luer-lock female connector and the luer-lock male connector comprise threads suitable for engaging with one another.

Furthermore, the luer-lock female connector and the luer-lock male connector have conical surfaces coming into contact with one another in order to establish a sealed fluidic connection between the first piece of equipment and the second piece of equipment, when the threads are engaged with one another.

The conical surface and/or the external thread of the luer-lock female connector of the first piece of equipment may however have imperfections which, as a consequence, compromise this sealing. Such imperfections can appear, in particular, in the case where the first piece of equipment is a vial obtained by a blow-fill-seal process.

### DISCLOSURE OF THE INVENTION

An aim of the present invention is to improve the fluidic connection seal between a male connector and a vial equipped with a female connector, when the female connector of the vial has imperfections at the threads or on the frustoconical sealing area.

This is the achieved by the kit as defined in claim 1.

In addition, the kit may have the features defined in the dependent claims, including the following features taken alone or in combination when this is technically possible.

In an embodiment, the first angle is between 25 degrees and 35 degrees with respect to the axis. In another embodiment, the first angle is between 10 degrees and 15 degrees with respect to the axis.

Preferably, the second angle is between 4 and 10 degrees with respect to the axis.

Preferably:
the first frustoconical portion has a first length measured parallel to the axis which is between 2 and 3 millimetres, and/or
the second frustoconical portion has a second length measured parallel to the axis which is between 6 millimetres and 8 millimetres.

Preferably, the sleeve has an inner surface in which the internal thread is formed, an outer surface opposite the inner surface, and a connecting surface connecting the inner surface to the outer surface, the connector furthermore comprising a fluid seal fixed on the connecting surface.

Preferably, the connector comprises two elements that can be gripped by a user, formed on the outer surface of the sleeve.

Preferably, the two grippable elements are two fins diametrically opposed with respect to the axis.

### DESCRIPTION OF THE FIGURES

Other features, aims and advantages of the invention will emerge from the following description, which is given purely by way of illustration and not being limiting and which should be read with reference to the attached drawings, in which:

FIG. 1 is a perspective view of an interconnector according to an embodiment.

FIG. 2 is a side view of the interconnector of FIG. 1.

FIG. 3 is a sectional view of the interconnector of FIG. 1.

FIG. 4 is a sectional view of a male connector constituting a part of the interconnector of FIG. 1.

FIG. 5 is a view from above of a vial according to an embodiment, in a closed state.

FIG. 6 is a side view of the vial of FIG. 5, in an open state.

FIG. 7 is a sectional view of the vial of FIG. 5.

FIG. 8 is a side view of an injection device according to an embodiment.

FIG. 9 is a sectional view of the injection device of FIG. 8.

FIG. 10 is a side view of two pieces of equipment forming an injection kit according to an embodiment in a disassembled state.

FIG. 11 is a side view of the kit of FIG. 10 in an assembled state.

FIG. 12 is a perspective view of an interconnector according to another embodiment.

FIG. 13 is a sectional view of the interconnector of FIG. 12.

In all figures, similar elements have identical reference signs.

### DETAILED DESCRIPTION OF THE EMBODIMENTS

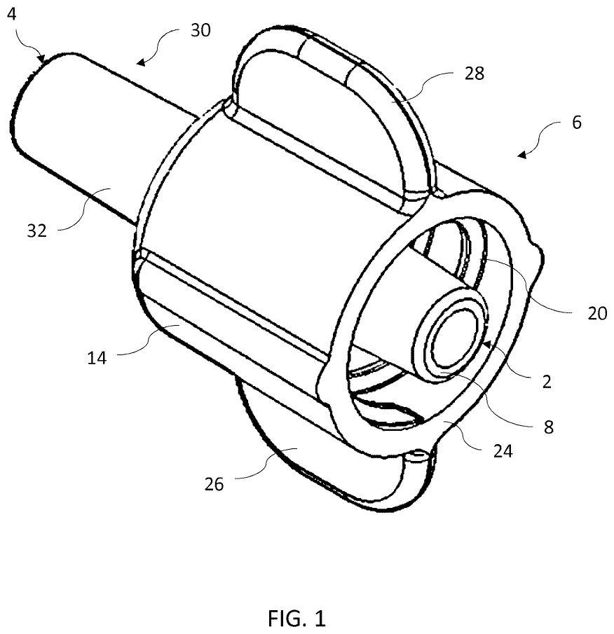

FIGS. 1 to 3 show an interconnector 1. The function of the interconnector 1 is to fluidically interconnect a first piece of equipment and a second piece of equipment, in other words to establish a fluidic connection enabling the transfer of a fluid from the first piece of equipment to the second piece of equipment. It will be seen below that these two pieces of equipment can respectively be a vial and an injection system for delivering a fluid contained in the vial.

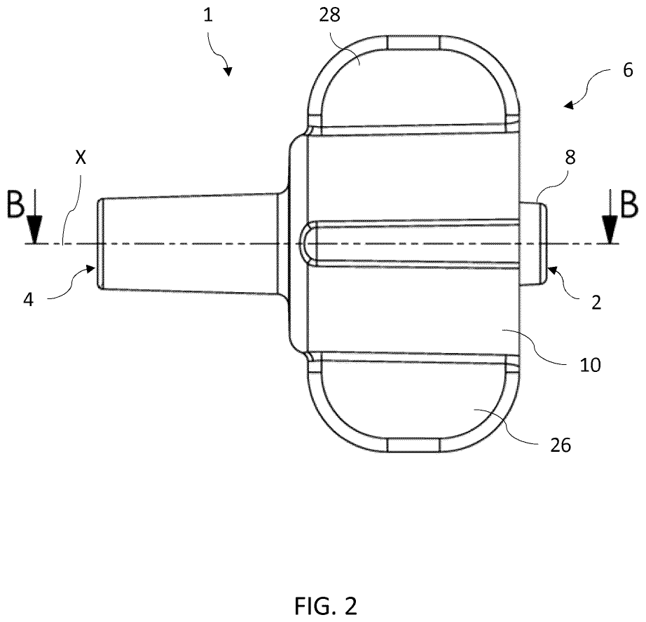

The interconnector 1 extends along an axis X from a first free end 2 to a second free end 4 opposite the first free end 2. The axis X is represented in FIG. 2 by a dashed line.

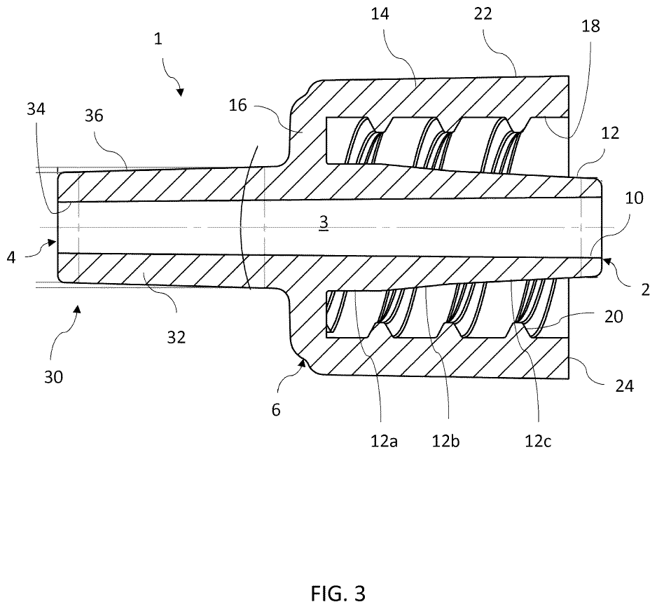

The interconnector 1 defines a through-passage forming such a fluidic connection. This passage, visible in FIG. 3, has a first access opening to the outside of the interconnector 1 at the first free end 2, and a second access likewise opening to the outside of the interconnector at the second free end 4. The passage 3 extends along the axis. The passage is straight.

The interconnector 1 comprises a first connector 6. The first connector 6 is a male connector suitable for being connected to the first piece of equipment, more precisely to a luer-lock female connector of the first piece of equipment.

The first connector 6 comprises an annular male part 8 terminating at the first free end 2 where the first access opens out.

The male part 8 has an inner surface 10 extending around the axis, so as to delimit a first portion of the above-mentioned passage, and delimiting, in particular, the first access. The inner surface is rotationally cylindrical around the axis X.

In addition, the male part 8 has an outer surface 12 opposite the inner surface 10. The outer surface extends around the inner surface 10.

In general, the outer surface 12 tapers towards the first free end 2.

The outer surface 12 comprises a rotationally cylindrical portion 12a around the axis X, a first frustoconical portion 12b extending the cylindrical portion 12a towards the first free end 2, and a second frustoconical portion 12c extending the first frustoconical part 12b towards the first free end 2.

The cylindrical portion 12a has a length, measured parallel to the axis X, of between 1 and 3 millimetres, for example 2 millimetres.

The cylindrical portion 12a has a constant diameter of between 3 and 5 millimetres, for example 4.35 millimetres with a tolerance of 0.05 millimetres.

The first frustoconical portion 12b has a length, measured parallel to the axis X, of between 2 and 4 millimetres, for example 2.5 millimetres.

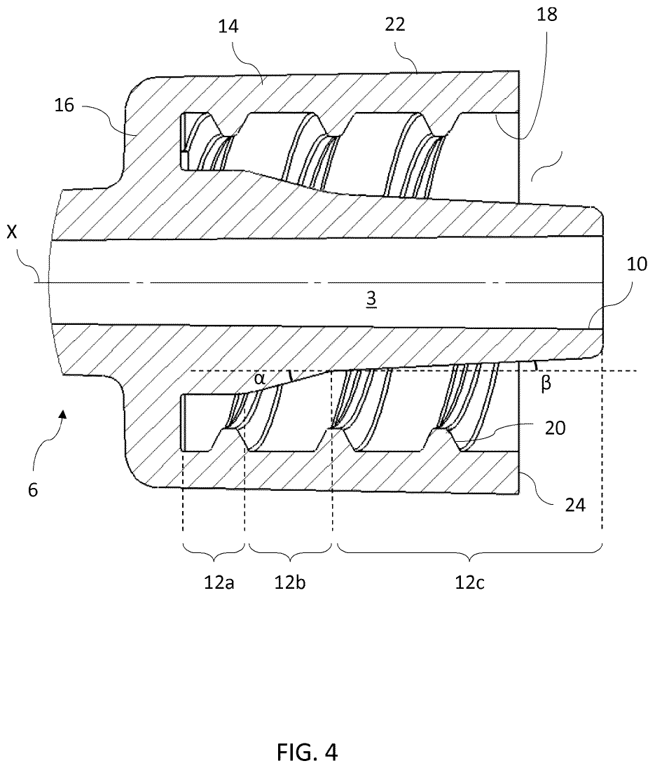

The first frustoconical portion 12b has a slope forming a first angle with respect to the axis X, as can be seen most particularly in FIG. 4.

In an embodiment, the first angle is between 25 degrees and 35 degrees with respect to the axis X, for example 30 degrees. In another embodiment, the first angle is between 10 degrees and 15 degrees with respect to the axis X, for example 12.5 degrees.

The second frustoconical portion 12c has a length, measured parallel to the axis X, of between 6 and 8 millimetres, for example 7 millimetres.

The second frustoconical portion 12c has a slope forming a second angle with respect to the axis X. The second angle is less than the first angle. This is equivalent to saying that the second frustoconical portion 12c has a conicity less than the conicity of the first frustoconical portion 12b or, more familiarly, that the second frustoconical portion 12c is less sloped than the first frustoconical portion 12b, with respect to the axis X.

The second angle is between 4 and 10 degrees with respect to the axis X, for example 7 degrees.

The outer surface 12 has a minimum diameter, at the free end 2, of between 3 and 4 millimetres, for example 3.61 millimetres with a tolerance of 0.05 millimetres.

The first connector 6 further comprises a sleeve 14 extending around and at a distance from the male part 8.

The sleeve 14 is fixed with respect to the male part 8. The sleeve 14 is connected to the male part 6 by an annular connection part 16 extending radially around the axis X.

The sleeve 14 has an inner surface 18 facing the outer surface 12 of the male part 8. Thus, the male part 8 and the sleeve 14 delimit between them an annular space suitable for receiving a part of the first above-mentioned piece of equipment (it will be seen in the following precisely that this part can be the neck of a vial).

The inner surface 18 has a maximum diameter between 7 and 9 millimetres, for example 8 millimetres with a tolerance of 0.1 millimetres.

The first connector 6 comprises an internal thread 20 which is formed in the inner surface 18. The internal thread 20 is suitable for cooperating with the external thread of a luer-lock female connector of the first piece of equipment, when the male part is inserted in such a luer-lock female connector.

The internal thread 20 has a pitch of approximately 2 millimetres.

The outer surface 22 of the sleeve 14 is rotationally cylindrical.

The outer surface 22 has a diameter between 9 and 11 millimetres, preferably 10 millimetres.

The sleeve 14 also has a connecting surface 24 connecting the inner surface to the outer surface. The connecting surface is annular. Although not illustrated, a seal, for example an o-ring, can optionally be fixed on this connecting surface 24 in order to improve the sealing of the connection between the first connector 6 and a luer-lock female connector of the first piece of equipment.

The sleeve 14 has a length, measured parallel to the axis X, of between 9 and 11 millimetres, for example 10.1 millimetres.

The first connector 6 further comprises two gripping elements 26, 28 formed on the outer surface 22 of the sleeve 14, in order to help a user to rotate the sleeve 14 around the axis X. The two gripping elements 26, 28 can be seen in FIGS. 1 and 2.

The two gripping elements 26, 28 form two fins projecting radially on the outer surface 22.

The two gripping elements 26, 28 are diametrically opposite with respect to the axis X.

The male part 8 projects outside the sleeve 14 in a direction parallel to the axis X. The length of the male part 8 along the axis X is greater than the length of the sleeve 14, measured in the same direction, so that the free end of the male part 8 is further away from the connection part 16 than is the connecting surface 24. In other words, the connecting surface 24 has a position along the axis X which is between the connection part 16 and the free end of the male part 8.

The interconnector 1 further comprises a second connector 30 suitable for being connected to a second piece of equipment (when the first connector 6 is itself connected to the first piece of equipment).

The second connector 30 comprises a second male part 32. The male part 32 is annular and terminated by the second free end 4 where the second access of the passage 3 opens out.

The second male part 32 has a second inner surface 34 extending around the axis X, so as to delimit a second portion of the above-mentioned passage 3 and delimiting, in particular, the second access located at the second free end 4. The second inner surface 34 is rotationally cylindrical around of the axis X.

The second inner surface 34 continuously extends the inner surface 10 of the male part 8 of the first connector 6.

The second male part 32 has, in addition, a second outer surface 36 opposite the second inner surface 34. The outer surface 36 extends around the inner surface 34.

The second outer surface 36 tapers towards the second free end 4.

The second outer surface 36 is frustoconical. The conicity of the second outer surface is preferably 6%. In this case, the second connector is a male luer connector.

The second connector 30 does not have a threaded sleeve, unlike the first connector 6.

The second connector 30 has a length, measured parallel to the axis X between the connection part 16 and the second free end 4, of between 8 and 9 millimetres, for example 8.45 millimetres.

The interconnector is made as a single piece. Preferably, the interconnector is made of a material such as polyethylene or polypropylene, or any material that is thermoformable by injection and compatible with a medical use.

### Vial

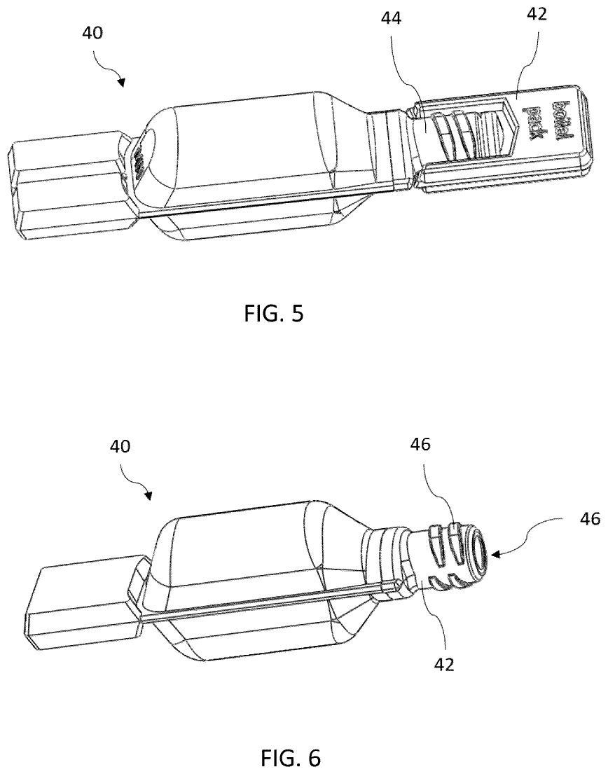

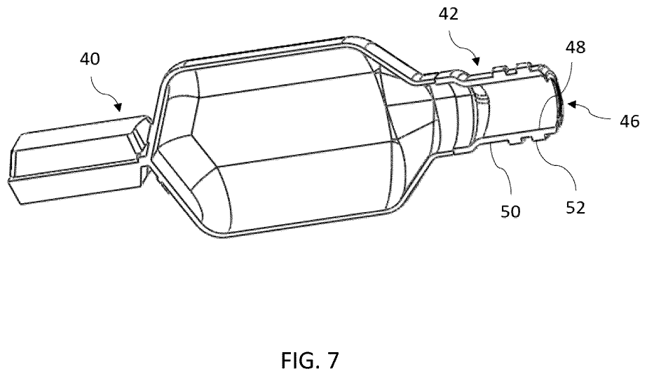

With reference to FIGS. 5 to 7, a first piece of equipment that can be connected to the first connector 6 of the interconnector is a vial 40.

The vial 40 is obtained by a blow-fill-seal process. In a variant, the vial 40 is obtained by other plastics processing techniques, such as injection, injection-blow moulding or extrusion blow-moulding.

The vial 40 is a single piece. Such a feature allows the vial to pose fewer problems for preserving asepsis during subsequent use, compared with vials comprising caps (needing to be sterilised).

The vial 40 is made of a deformable material, preferably an elastomer. Very preferably, this material is low-density polyethylene, which is a material having the advantage of being particularly flexible.

The deformable material allows, in particular, a user to press the vial 40 in order to expel a fluid that it contains.

The vial 40 comprises a neck 42. The neck 42 is sealed by a sealing part 44: the vial is therefore in a closed state, as can be seen in FIG. 5. The sealing part 44 is suitable for being broken by a user, for example through a rotary movement, and thus for being detached from the remainder of the vial 42 so as to form an opening 46 at the neck 42 giving access to the inside of the vial 40 from the outside of the vial 40. The vial is then in an open state, such as that shown in FIG. 6.

The neck 42 has an inner surface 48 delimiting a passage between the inside and the outside of the vial terminating by the opening 46, and an outer surface 50 opposite the inner surface.

The inner surface 48 is frustoconical or cylindrical.

The vial 40 further comprises an external thread 52. The external thread 52 is formed in the outer surface 50 of the neck. The external thread 52 is suitable for cooperating with the internal thread 20 of the interconnector 1.

The inner surface 48 and the external thread 52 form a female connector when the vial is in its open state. In reality, the neck constitutes a female element suitable for receiving a male part of a luer-lock male connector or the male part 8 of the first connector 6.

The vial can be used to store a fluid. The fluid may comprise a vaccine or a sterile product that is able to be delivered in the form of injection, mist or drops.

### Injection Device With Female Connector

A second piece of equipment that is able to be connected to the second connector 30 of the interconnector is a fluid injection device (not illustrated).

The injection system comprises a hollow needle and a connector suitable for being connected to the second connector 30 of the interconnector 1.

The hollow needle has a bevelled free end. An outlet is formed at this free end, via which a fluid can be delivered.

The injection system delimits an internal passage terminating at the outlet. This internal passage is in fluidic communication with the internal passage of the interconnector, when the connector of the injection system is connected to the interconnector.

The injection system connector is a female connector, preferably a female luer connector. For this purpose, the female connector has an inner surface delimiting the above-mentioned passage, and having a conicity of order 6%.

### Injection Kit With Interconnector

The interconnector 1 can be combined with a first piece of equipment and a second piece of equipment in order to form a kit. In particular, this kit can be used as follows when the first piece of equipment is the previously-described vial 30, and the second piece of equipment is the previously-described injection system with female connector.

It is assumed that in its closed state the vial contains a fluid to be administered, for example a fluid comprising a vaccine.

A user breaks the sealing part 42 of the vial 40 by rotating it relative to the rest of the vial 40, thus opening the vial at the neck 44.

The user connects the vial 40 to the interconnector 1. For this purpose, the user inserts the male part 8 of the first connector 6 of the interconnector 1 into the neck 42 of the vial 40, so that the first access to the passage 3, formed at the free end 2 of the interconnector 1, opens out into the vial. During this insertion, the internal thread 20 of the first connector 6 comes into contact with the external thread formed on the neck of the vial. The user rotates the interconnector 1 around its axis X with respect to the vial such that the two threads are engaged by screwing. At a certain stage in the screwing, the outer surface 12 of the male part 8 comes into contact with the inner surface of the neck of the vial. The presence of the two portions 12b and 12c in the outer surface 12, having different conicity, ensures a good seal between the inside of the vial and the internal passage 3 which passes through the interconnector 1, and this even if, in its open state, the vial has geometric imperfections.

The user further connects the interconnector 1 to the injection system. For this purpose, the user inserts the male part 32 of the second connector 30 of the interconnector 1 into the female luer connector of the injection system.

The two preceding steps can be carried out in any order.

Once these two steps are carried out, the user obtains a device in which a fluid connection can be established between the inside of the vial and the outlet formed at the end of the injection needle, this fluidic connection comprising, in particular, the passage 3 passing through the interconnector 1.

In order to expel the fluid initially contained in the vial, the user can press two opposite sides of the vial. The fluid passes through the neck, penetrates into the interconnector 1 via the first access located at the first free end 2, passes through the internal passage 3 of the interconnector 1, exits via the second access located at the second free end 4, then penetrates into the injection system before exiting via the opening formed at the end of the needle.

The interconnector 1 has the advantage of enabling a fluidic connection to be established between the injection system and the vial, without having to modify the respective structures of these two pieces of equipment. In particular, the double conicity of the first connector 6 ensures that the fluidic connection established between these two pieces of equipment is well sealed.

### Injection Kit Without Interconnector

In the kit as described above, a connector 6 with double conicity is incorporated in the interconnector 1 in order to improve the fluidic sealing of the device resulting from the connection of the interconnector 1 to the first piece of equipment (vial 30) and to the second piece of equipment (injection system).

It is possible to incorporate a connector with double conicity having the same features as the first connector 6 in pieces of equipment other than an interconnector in order to obtain this fluidic sealing improvement.

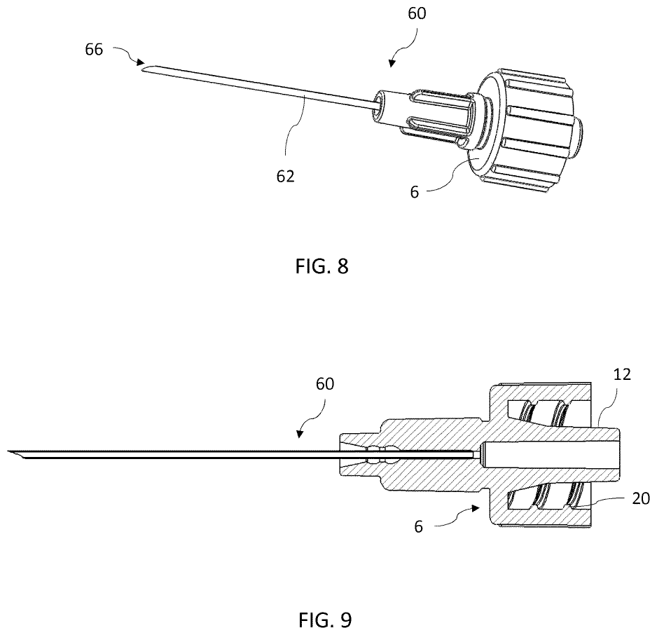

In particular, it is possible to include a connector with double conicity having the same features as the first connector 6 in an injection system 60 according to a second embodiment shown in FIGS. 8 and 9. The injection system 60 according to this second embodiment differs from the injection as previously described, by the fact that its connector is not a female luer connector, but the connector 6 as previously described (including, in particular, the outer surface 12 with double conicity, as well as the internal thread 20).

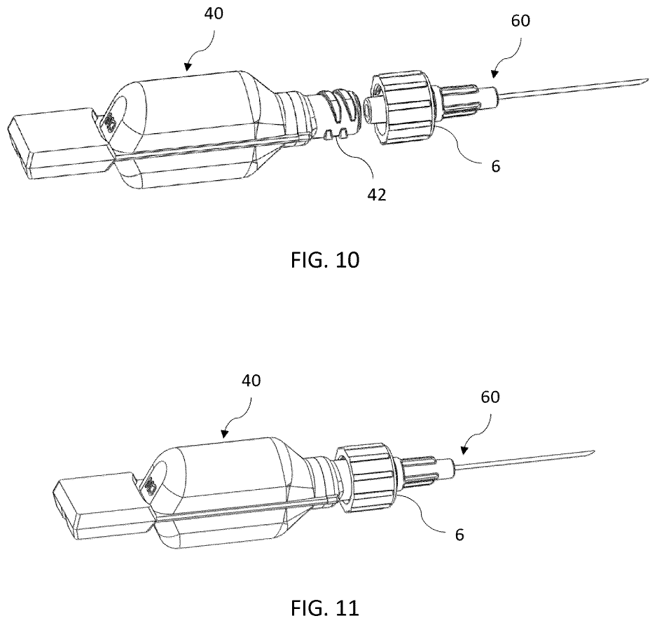

The injection system 60 according to this second embodiment can be connected directly to the previously described vial 40, without requiring an interconnector between the two, as shown in FIGS. 9 and 10. In other words, the injection system according to this second embodiment forms, with the vial 40, a second kit not requiring an interconnector. For this purpose, the user inserts the male part 8 of the first connector 6 of the injection system 60 in the neck 42 of the vial 40, so that the first access to the passage 3, formed at the free end 2 of the interconnector 1, opens out into the vial. During this insertion, the internal thread 20 of the connector 6 comes into contact with the external thread 52 formed on the neck 42 of the vial 40. The user rotates the injection system 1 with respect to the vial 40 such that the two threads 20, 52 are engaged by screwing. At a certain stage in the screwing, the outer surface 12 of the male part 8 comes into contact with the inner surface of the neck of the vial.

### Other Kits for Delivering a Fluid

In the above, two kits were proposed in which the vial 30 is placed in fluidic communication with injection devices provided with a needle. However, the vial 40 can be combined with other devices for delivering a fluid, in particular a spray device. The spray device comprises a spray nozzle which can deliver a fluid emanating from the vial in the form of a nasal spray for delivering a medication or nasal vaccine. Of course, the spray device can comprise the connector 6 directly connectable to the vial 40, or else a female connector connectable to the second connector 30 of the interconnector 1.

Alternatively, it is possible to combine the vial 40 with a device for delivering a fluid comprising a sterilising filter with a porosity less than or equal to 0.22 microns. This type of filter is suitable for delivering a fluid in the form of drops, such as a sterile medication. The filter can comprise the connector 6 directly connectable to the vial 40, or else a female connector connectable to the second connector 30 of the interconnector 1. The system obtained with the vial and the device comprising the filter (and where appropriate with the interconnector 1) is a multi-dose system that can be used over several days of several weeks.

### Other Male Connector Embodiments

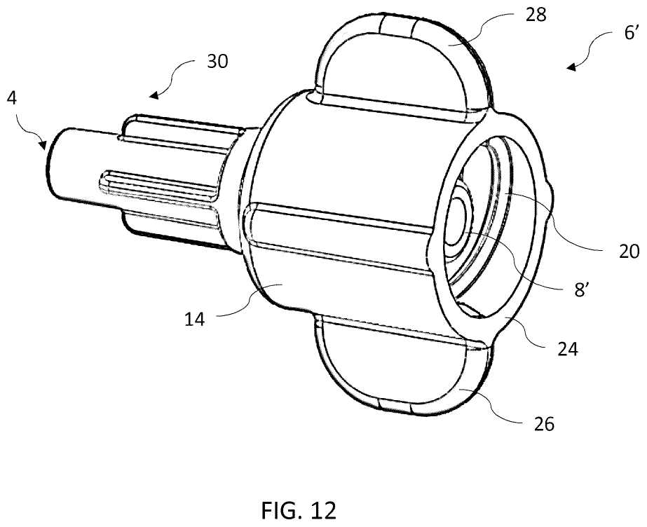

FIG. 12 shows a male connector 6′, which differs from the male connector 6 through the following features (all other things being equal).

The male connector 6′ comprises a male part 8′ identical to the male part 8 except that the free end of the male part 8′ is set back with respect to the sleeve 14 in a direction parallel to the axis X. The term “set back” shall mean that the free end of the male part 8′ does not project out of the sleeve. On the contrary, this free end is located inside the sleeve at a non-zero distance from the free end of the sleeve 14, this distance being measured parallel to the axis X.

With reference to FIG. 10, the male connector 6′ comprises an annular seal 17 fixed on the connection part 16. This seal is arranged in the space provided between the male part 8′ and the sleeve 14, such that the neck 42 of the vial comes into contact with the seal when the male part 8 is inserted in the female connector formed by the neck 42.

The seal is arranged at a distance from the internal thread 20 in a direction parallel to the axis X. Thus, the external thread 20 does not extend up to the connection part 16, which limits the travel distance of the neck towards the seal. This arrangement can prevent ovalisation of the seal which would be caused by excessive crushing of the seal. This ovalisation gives the seal an oval shape when it is observed in a direction parallel to the axis X, instead of taking a circular shape. This oval shape compromises the sealing performance of the seal.

The seal of the male connector 6′ is optional. It can be incorporated in the male connector 6 previously discussed.

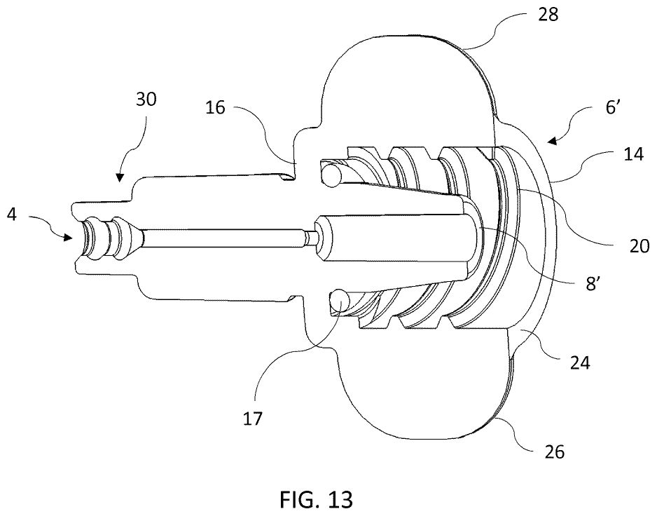

The male connector shown in FIGS. 12-13 is part of an interconnector, but could alternatively be part of another type of device to be connected to the vial.

1. A kit comprising a vial and a male connector, wherein the vial is obtained by a blow-fill-seal process, and comprises:
a neck comprising an external thread,
a sealing part which seals the neck, the sealing part being breakable by a user so as to reveal an opening delimited by the neck giving access to the inside of the vial from the outside of the vial,
and wherein the male connector comprises:
a male part suitable for being inserted in the female connector after the sealing part has been broken, the male part having an outer surface extending around an axis and tapering towards a free end of the male part,
a sleeve extending around and at a distance from the male part, the sleeve comprising an internal thread suitable for cooperating with the external thread when the male part is inserted in the female connector, wherein the outer surface comprises a first frustoconical portion forming a first angle with respect to the axis, and a second frustoconical portion extending the first frustoconical portion towards the free end, the second frustoconical portion forming a second angle with respect to the axis, the second angle being less than the first angle.

2. The kit according to claim 1, wherein the first angle is between 25 degrees and 35 degrees with respect to the axis.

3. The kit according to claim 1, wherein the first angle is between 10 degrees and 15 degrees with respect to the axis.

4. The kit according to claim 1, wherein the second angle is between 4 and 10 degrees with respect to the axis.

5. The kit according to claim 1, wherein:
the first frustoconical portion has a first length measured parallel to the axis which is between 2 and 3 millimetres, or
the second frustoconical portion has a second length measured parallel to the axis which is between 6 millimetres and 8 millimetres.

6. The kit according to claim 1, wherein the sleeve has an inner surface in which the internal thread is formed, an outer surface opposite the inner surface, and a connecting surface connecting the inner surface to the outer surface, the connector further comprising a fluid seal fixed on the connecting surface.

7. The kit according to claim 1, wherein the male connector comprises two elements which can be gripped by a user, formed on the outer surface of the sleeve.

8. The kit according to claim 7, wherein the two elements are two fins that are diametrically opposed with respect to the axis.

9. The kit according to claim 1, comprising a device, the device comprising the male connector and being suitable for delivering a fluid coming from the vial, when the male connector is connected to the female connector.

10. The kit according to claim 9, wherein the device comprises an injection needle for delivering the fluid, a spray nozzle for delivering the fluid in the form of a spray, or a sterilising filter enabling the fluid to be delivered in the form of drops.

11. The kit according to claim 1, comprising an interconnector, the interconnector comprising: the male connector, a second connector, and a passage capable of forming a fluid connection between the vial and another piece of equipment when the male connector is connected to the vial while the second connector is connected to the other piece of equipment.

12. The kit according claim 1, further comprising a syringe comprising a male luer connector suitable for being connected to the female connector, the syringe being suitable for drawing a fluid from the vial when the male luer connector is connected to the female connector.

13. The kit according to claim 1, wherein the male connector comprises:
an annular connection part extending radially around the axis in order to connect the male element to the sleeve,
a seal fixed on the annular connection part and arranged between the male part and the sleeve, such that the neck comes into contact with the seal when the male part is inserted in the female connector, the seal being at a distance from the internal thread in a direction parallel to the axis.

14. The kit according to claim 1, wherein the free end of the male part is set back with respect to the sleeve in a direction parallel to the axis.

15. The kit according to claim 1, wherein the vial is made of low-density polyethylene.
